(19) United States
(12) Patent Application Publication
(10) Pub. No.: US 2016/0220734 A1
(43) Pub. Date: Aug. 4, 2016

(54) PHOTO-ACTIVE AND RADIO-OPAQUE SHAPE MEMORY POLYMER-GOLD NANOCOMPOSITE MATERIALS FOR TRANS-CATHETER MEDICAL DEVICES

(71) Applicant: THE REGENTS OF THE UNIVERSITY OF COLORADO, A BODY CORPORATE, Denver, CO (US)

(72) Inventors: Kiran Dyamenahalli, Denver, CO (US); Robin Shandas, Boulder, CO (US)

(21) Appl. No.: 15/024,394

(22) PCT Filed: Oct. 2, 2014
(86) PCT No.: PCT/US14/58916

§ 371 Date: Mar. 24, 2016

Related U.S. Application Data
(60) Provisional application No. 61/886,047, filed on Oct. 2, 2013.

(51) Int. Cl. A61L 29/12; A61L 29/18; A61L 31/12; A61B 17/12; A61L 31/18; A61L 27/44; A61L 27/50; A61K 49/04; A61L 29/14; A61L 31/14

(52) U.S. Cl. CPC A61L 29/126; A61L 29/14; A61L 29/18; A61L 31/128; A61L 31/14; A61L 31/18; A61L 27/446; A61L 27/50; A61K 49/04; A61B 17/12113; A61B 17/1214; A61L 2400/16; A61L 2400/12; A61F 2/86

(57) ABSTRACT

There is disclosed a trans-catheter cardiovascular device, comprising a composite material having gold nanoparticles embedded in a shape memory polymer. In an embodiment, the gold nanoparticles are surface-functionalized gold nanoparticles. In an embodiment, shape memory polymer is a cross-linked shape memory polymer. In various embodiments, the shape memory polymer forms one of a stent, an embolic coil, a venous filter, a vascular graft, and a cardiac septal defect closure device. Other embodiments are also disclosed.

### REFERENCE TO PENDING PRIOR PATENT APPLICATION

This application claims the benefit under 35 U.S.C. 119 (e) of U.S. Provisional Patent Application No. 61/886,047, filed Oct. 2, 2013 by Kiran Dyamenahalli et al. for “PHOTO-ACTIVE AND RADIO-OPAQUE SHAPE MEMORY POLYMER—GOLD NANOCOMPOSITE MATERIALS FOR TRANS-CATHETER MEDICAL DEVICES”, which patent application is hereby incorporated herein by reference.

### BACKGROUND

Percutaneous intervention with trans-catheter devices is based on the principle that lesions in the cardiovascular system can be accessed and repaired from inside the heart and vascular compartment, without the need for open surgical procedures. Its application has grown significantly over the last two decades and trans-catheter cardiovascular devices (TCDs) now include coils and particulates for embolization of vascular malformations (especially aneurysms) and arterial tumor supplies, patches and grafts for cardiac septal defect closure or vascular reconstruction, coronary and peripheral artery stents, and filters designed to catch blood clots before they reach the lungs or carotid arteries (reducing the risk for pulmonary embolism or stroke, respectively). Sokolowski W, Metcalfe A, Hayashi S, Yahia L, Raymond J., Medical applications of shape memory polymers. Biomedical materials (Bristol, England). 2007;2(1):S23-7. Available at: http://www.ncbi.nlm.nih.gov/pubmed/18458416. Accessed Aug. 20, 2010; Yakacki C M, Lyons M B, Rech B, Gall K, Shandas R., Cytotoxicity and thermomechanical behavior of biomedical shape-memory polymer networks post-sterilization. Biomedical materials (Bristol, England). 2008;3 (1):015010. Available at: http://www.ncbi.nlm.nih.gov/pubmed/18458497. Accessed Aug. 20, 2010; Small W, Singhal P, Wilson T S, Maitland D J., Biomedical applications of thermally activated shape memory polymers. Journal of materials chemistry. 2010;20(18):3356-3366. Available at: http://www.pubmedcentral.nih.gov/articlerender.fcgi?artid=3023912&tool=pmcentrez&rendertype=abstract]. Percutaneous intervention is often a good option in patients for whom customary vascular or trans-thoracic cardiac surgery is contraindicated, due to heart failure, previous surgeries, or age. Moreover, the initial promises of this approach, including shortened procedure and recovery times, reduced costs and repeat procedure rates, and improved patient outcomes, have been achieved in many cases. Iribarne A, Easterwood R, Chan E Y H, et al., The golden age of minimally invasive cardiothoracic surgery: current and future perspectives. Future Cardiology. 2012;7(3):333-346.]

However, the adoption of TCDs has been slowed by recent reports which fail to show equivalent or superior clinical outcomes compared to traditional surgical repairs, narrow indication windows for certain devices, issues with biocompatibility, and related post-procedure complications. Many of these complications, such as recanalization and bleeding of coiled aneurysms or stent-associated thrombus formation, are caused by failures in either the design or composition of TCDs. Tan I Y L, Agid R F, Willinsky R., Recanalization rates after endovascular coil embolization in a cohort of matched ruptured and unruptured cerebral aneurysms. Interventional neuroradiology: journal of peritherapeutic neuroradiology, surgical procedures and related neurosciences. 2011; 17(1):27-35. Available at: http://www.pubmedcentral.nih.gov/articlerender.fcgi?artid=3278032&tool=pmcentrez&rendertype=abstract.] Accordingly, their resolution drives research and development to attempt to solve fundamental materials science problems. Conventional materials used in most TCDs include platinum, stainless steel, titanium, nickel, iridium, cobalt, molybdenum, tantalum, chromium, and their alloys (e.g. Nitinol, an alloy of titanium and nickel). Metals are used because they are durable and generally visible using X-ray based imaging modalities.

However, their mechanical properties, such as flexural stiffness and compressibility, are only tunable within a very narrow range and their surface properties, which determine their tendency to incite an inflammatory response or form blood clots, are largely static. Additionally, metals are unable to elute drugs without a polymer coating or biodegrade once their function is complete, and they distort computed tomography (CT) and magnetic resonance imaging (MRI) scans through significant artifact generation. In some cases, placement of ferromagnetic metal TCDs is a contraindication to MRI scans due to its use of high magnetic field strengths. Finally, in terms of both material and processing costs, metal components can be very expensive to manufacture, transferring higher costs to patients and providers. Hampikian J, Heaton B, Tong F, Zhang Z, Wong C P., Mechanical and radiographic properties of a shape memory polymer composite for intracranial aneurysm coils. Materials Science and Engineering: C. 2006;26(8):1373-1379.]

The shift toward percutaneous interventions and importance of material properties in TCD performance is perhaps best illustrated by a specific example. In particular, here, the case of embolic coils for aneurysm repair is taken a bit further. Aneurysms are pathologically-weakened and dilated sections of blood vessels that are at increased risk of rupture. In the cerebral vasculature, rupture of an aneurysm can lead to catastrophic hemorrhagic stroke. One option for early intervention is a major neurosurgical procedure involving a craniotomy and placement of a clip at the neck of the malformation. However, this approach has lost ground to minimally-invasive embolization techniques, which involve trans-catheter delivery of small coils into the aneurysm, producing an effective seal through physical packing, hemostasis, thrombosis, and eventually neointimal formation. To date, embolic coils have primarily been fabricated using stainless steel or platinum. Although such coils are well-accepted clinically, they are limited by their poor capacity for shape-memory, poor resistance to kinking, and relatively high stiffness, all of which prevent optimal packing of the aneurysm. Furthermore, CT and MRI artifacts generated by metal coils prevent accurate visualization of proximal anatomy. As such, clinicians are typically obligated to use fluoroscopy for follow-up evaluation, increasing radiation dose to the patient.

In view of the drawbacks of metals, the prospect of fully-polymeric TCDs have sparked much interest. Synthetic polymers offer a far more attractive palette of features, including reduced device costs, decreased or absent MRI and CT imaging artifacts, and the ability to tune stiffness, surface interactions with blood components, biodegradation, and drug elution. Among polymers, shape memory polymers (SMPs) have highly desirable properties for catheter-based storage and release. These materials can recover almost any pre-determined shape of very low stiffness after being heated above a tunable glass-transition temperature (T_{g}). Sokolowski W, Metcalfe A, Hayashi S, Yahia L, Raymond J., Medical applications of shape memory polymers. Biomedical materials (Bristol, England). 2007;2(1):523-7; Baer G M, Wilson T S, Small W, et al., Thermomechanical properties, collapse pressure, and expansion of shape memory polymer neurovascular stent prototypes. Journal of biomedical materials research. Part B, Applied biomaterials. 2009;90(1):421-9. Available at: http://www.ncbi.nlm.nih.gov/pubmed/19107804. Accessed Jan. 2, 2011; Yakacki C M, Shandas R, Lanning C, et al., Unconstrained recovery characterization of shape-memory polymer networks for cardiovascular applications. Biomaterials. 2007;28(14):2255-63. Available at: http://www.ncbi.nlm.nih.gov/pubmed/17296222.] Repeated strain recoveries of several hundred percent are possible over multiple cycles. Yakacki C M, Willis S, Luders C, Gall K., Deformation Limits in Shape-Memory Polymers. Advanced Engineering Materials. 2008;10(1-2):112-119. Available at: http://doi.wiley.com/10.1002/adem.200700184. Accessed Aug. 20, 2010; Nair D P, Cramer N B, Scott T F, Bowman C N, Shandas R., Photopolymerized Thiol-Ene Systems as Shape Memory Polymers. Polymer. 2010;51(19):4383-4389. Available at: http://linkinghub.elsevier.com/retrieve/pii/S0032386110006403. Accessed Jul. 29, 2010.] This strain-recovery feature could allow, for instance, embolic polymer devices to recover a helical coil conformation upon release at body temperature, after being stored in a catheter in a compacted or elongated form. Heaton B, (Georgia IOT) A Shape Memory Polymer for Intracranial Aneurysm Coils: An Investigation of Mechanical and Radiographic Properties of a Tantalum-Filled Shape Memory Polymer Composite. 2004; Baer G M, Small W, Wilson T S, et al. Fabrication and in vitro deployment of a laser-activated shape memory polymer vascular stent. Biomedical engineering online. 2007;6:43. Available at: http://www.ncbi.nlm.nih.gov/pubmed/18042294.]

Even so, SMPs do have drawbacks. For instance, native SMPs generally still do not offer enough flexibility to span the broad range in bulk mechanical properties desired to fabricate ideal TCDs. Moreover, in any TCD application, accurate placement is critical to device performance and safety, and even with the advent of real-time/4D MRI, X-ray based imaging modalities are almost always employed in this capacity. Spahn M., Flat detectors and their clinical applications. European radiology. 2005;15(9):1934-47. Available at: http://www.ncbi.nlm.nih.gov/pubmed/15806363. Accessed Aug. 20, 2011; Ghaye B, Dondelinger R F. Imaging guided thoracic interventions. European Respiratory Journal. 2001:507-528.] Polymers, though, are largely radiolucent, so heavy-element fillers are often used to absorb and scatter X-rays. Salamone J., Radiopaque Polymers. Polymeric materials encyclopedia: Q-S. 1996:7346-7350; Moszner N, Salz U., New Developments of Polymeric Dental Composites. Progress in Polymer Science. 2001;26(1):535-576.] For instance, barium sulfate, zirconium oxide and tantalum have been used in the orthopedic field for bone cement, Behl M, Razzaq M Y, Lendlein A., Multifunctional shape-memory polymers. Advanced materials (Deerfield Beach, Fla.). 2010;22(31):3388-410. Available at: http://www.ncbi.nlm.nih.gov/pubmed/20574951. Accessed Aug. 4, 2011; Bohner M., Design of ceramic-based cements and putties for bone graft substitution. European cells & materials. 2010;20:1-12. Available at: http://www.ncbi.nlm.nih.gov/pubmed/20574942; Lye K W, Tideman H, Merkx M a W, Jansen J, Bone cements and their potential use in a mandibular endoprosthesis. Tissue engineering. Part B, Reviews. 2009;15(4):485-96. Available at: http://www.pubmedcentral.nih.gov/articlerender.fcgi?artid=3113466&tool=pmcentrez&rendertype=abstract.] tantalum-filled SMPs have been evaluated in the research setting as embolic coil materials; Heaton B, (Georgia IOT) A Shape Memory Polymer for Intracranial Aneurysm Coils: An Investigation of Mechanical and Radiographic Properties of a Tantalum-Filled Shape Memory Polymer Composite. 2004] and iodinated monomers have been incorporated into denture base resins. Davy K W, Anseau M R, Berry C., Iodinated methacrylate copolymers as X-ray opaque denture base acrylics. Journal of dentistry. 1997;25(6):499-505. Available at: http://www.ncbi.nlm.nih.gov/pubmed/9604581]

However, the addition of these sorts of heavy-element fillers tend to disturb the original SMP characteristics. To overcome these issues, a special interest has developed in gold nanoparticles (GNPs) for generating radio-opaque and mechanically-optimal SMP nanocomposite materials. GNPs are advantageous for a number of reasons. First, compared to iodine, barium, tantalum and zirconium, gold offers a higher atomic weight (˜197) [21. Coursey J S, Schwab D J, Tsai J J, Dragoset R A. Atomic weights and isotopic compositions. NIST Physical Measurement Laboratory. 2010. Available at: http://www.nist.gov/pml/data/comp.cfm. Accessed Nov. 24, 2011.] and a superior ability to attenuate X-rays at most energy levels in the diagnostic range. [22. Hubbell J H, Seltzer S M. Tables of X-ray mass attenuation coefficients and mass energy-absorption coefficients. NIST Physical Measurement Laboratory. 2010. Available at: http://www.nist.gov/pml/data/xraycoef/index.cfm. Accessed Nov. 24, 2011] Moreover, due to gold's high K-shell binding energy, it can be imaged at even higher X-ray energies (>80 keV) for which bone and soft-tissue absorption are minimized, improving contrast and reducing ionizing radiation dose to the patient (as shown in present FIG. 1). Hainfeld J F, Slatkin D N, Focella T M, Smilowitz H M. Gold nanoparticles: a new X-ray contrast agent. British Journal of Radiology. 2006;79:248-253.] FIG. 1, in particular, plots NIST-published mass attenuation coefficient as a function of X-ray energy for gold, iodine and soft tissue. It is believed that the higher K-edge of gold, compared to iodine, should result in excellent contrast at high X-ray energies, for which tissue radiation dose is lower. Polymer-gold nanocomposites have primarily found use in optical applications, such as lenses, filters and light-emitting diodes, Balazs A C, Emrick T, Russell T P. Nanoparticle polymer composites: where two small worlds meet. Science (New York, N.Y.). 2006;314(5802):1107-10. Available at: http://www.ncbi.nlm.nih.gov/pubmed/17110567; Park J H, Lim Y T, Park O O, et al. Polymer/Gold Nanoparticle Nanocomposite Light-Emitting Diodes: Enhancement of Electroluminescence Stability and Quantum Efficiency of Blue-Light-Emitting Polymers. Chemistry of Materials. 2004;16(4):688-692. Available at: http://pubs.acs.org/doi/abs/10.1021/cm0304142] but in colloid form, functionalized GNPs have been evaluated as X-ray contrast agents for vasculature and micro-damaged bone, with favorable results. Hainfeld J F, Slatkin D N, Focella T M, Smilowitz H M. Gold nanoparticles: a new X-ray contrast agent. British Journal of Radiology. 2006;79:248-253; Alric C, Taleb J, Duc G L, et al., Gadolinium Chelate Coated Gold Nanoparticles as Contrast Agents for Both X-ray Computed Tomography and Magnetic Resonance Imaging. Journal of the American Chemical Society. 2008;130(13):5908-5915. Zhang Z, Ross R D, Roeder R K., Preparation of functionalized gold nanoparticles as a targeted X-ray contrast agent for damaged bone tissue. Nanoscale. 2010;2(4):582-6. Available at: http://www.ncbi.nlm.nih.gov/pubmed/20644762. Accessed Apr. 22, 2011]

The literature alludes to further potential benefits of GNPs, in addition to the first one discussed above. Second, by varying their size, aspect ratio, concentration, and surface chemistry, the bulk properties of the resulting composite material can be tailored to a very fine degree. Third, GNPs are very well characterized as being well-known for the ease and flexibility of their synthesis, excellent size and shape control, their long-term stability in a variety of solvents, and amenity to surface modification with thiols, amines and phosphines for dispersion in polymer environments. Giljohann D a, Seferos D S, Daniel W L, et al., Gold nanoparticles for biology and medicine. Angewandte Chemie (International ed. in English). 2010;49(19):3280-94. Available at: http://wmww.ncbi.nlm.nih.gov/pubmed/20401880; Pissuwan D, Niidome T, Cortie M B. The forthcoming applications of gold nanoparticles in drug and gene delivery systems. Journal of controlled release: official journal of the Controlled Release Society. 2009;149(1):65-71. Available at: http://www.ncbi.nlm.nih.gov/pubmed/20004222; Rotello V M., Drug and Gene Delivery using Gold Nanoparticles. Drug Delivery. 2007:40-45; Connor E E, Mwamuka J, Gole A, Murphy C J, Wyatt M D, Gold nanoparticles are taken up by human cells but do not cause acute cytotoxicity. Small (Weinheim an der Bergstrasse, Germany). 2005;1(3):325-7. Available at: http://www.ncbi.nlm.nih.gov/pubmed/17193451. Accessed Sep. 6, 2010.

Fourth, GNPs can confer entirely new properties to SMPs. Native polymers are electrical and thermal insulators, but GNPs may allow these properties to be controlled in a concentration-dependent manner. Likewise, GNPs can dissipate visible light as heat, allowing for indirect spatial control of thermal transitions (and thus shape) in SMP-GNP composites. Zhang et al. recently harnessed the unique surface plasmon resonance-enhanced absorption of green light to trigger shape changes in SMPs. Zhang H, Xia H, Zhao Y. Optically triggered and spatially controllable shape-memory polymer-gold nanoparticle composite materials. Journal of Materials Chemistry. 2011;Published.]

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In various exemplary embodiments, there is provided a customizable and thermally-responsive SMP-gold nanocomposite material for the design of next-generation TCDs. This material may preserve the best features of both metals and polymers, while adding properties that could allow for new modes of device delivery and use.

In various exemplary embodiments, a composite material is disclosed that includes surface-functionalized gold nanoparticles (GNPs) embedded in cross-linked shape memory polymers (SMPs). This material has a number of properties that may make it appropriate for the design of trans-catheter medical devices, including stents, embolic coils, venous filters, vascular grafts, cardiac septal defect closure devices, etc. These properties may include:
The ability to recover large strains (alkanethiol-functionalized gold nanoparticles improve the shape-recovery behavior of standard acrylate SMPs)
Several modifiable variables that provide control of the mechanical and thermo-mechanical properties of the material, including chemical composition, cross-linker molecular weight and density, and GNP size, surface chemistry and concentration.
Radio-opacity (X-ray visibility)
Minimal MRI and CT imaging artifacts
Absence of toxic components
Control of biodegradation
Photo-activation using green laser light (i.e., control of thermal transitions using GNPs to dissipate visible light as heat)
Enhanced thermal conduction compared with unmodified acrylate SMP
Enhanced ultrasound contrast compared with unmodified acrylate SMP
Low material and processing costs compared to typical metals used to fabricate trans-catheter devices

In various embodiments, GNPs may be used to modify the imaging properties of trans-catheter devices and the ability to control shape recovery and device release with visible light. While GNPs have been used as vascular X-ray contrast agents, and gold marker bands are used in some trans-catheter devices, efforts are unknown to use GNPs to confer radio-opacity to solid medical devices. Photo-activation with green laser light has been shown in non-acrylate SMPs (Zhang et al.), but no biomedical applications were discussed. A fiber-optic catheter carrying green light could provide precise spatial and temporal control of shape-recovery and device release or recovery. In addition, heat generation by GNPs upon exposure to green laser light may also allow for control of material polymerization in the presence of thermal initiators, which also has not been shown in the literature.

Additional objects, advantages and novel features of the technology will be set forth in part in the description which follows, and in part will become more apparent to those skilled in the art upon examination of the following, or may be learned from practice of the technology. The following figures help to illustrate exemplary embodiments of the structure and properties of the SMP-GNP composite system.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 is a plot of NIST-published mass attenuation coefficient as a function of X-ray energy for gold, iodine and soft tissue.

FIGS. 2A-2C are schematic illustrations of the general chemical structure of monomers used: tert-Butyl acrylate (tBA) (FIG. A), and poly(ethylene glycol)dimethacrylate (PEGDMA) 550 Mn (FIG. B); and polymer cross-linking with a gold nanoparticle (FIG. C).

FIG. 3 is a graph of the X-ray photoelectron spectrum of a DDT-functionalized gold nanoparticle surface, formed in accordance with the present system.

FIGS. 4A-4E are a series of data plots and transmission electron microscope (TEM) micrographs that together help to characterize DDT-functionalized gold nanoparticles. FIG. 4A is a plot of the UV-Vis spectrum, and FIG. 4B is graph of dynamic light scattering data of the gold nanoparticles. FIG. 4C is a transmission electron micrographs of DDT-functionalized gold nanoparticles dispersed in hexane; and FIGS. 4D and 4E are TEM micrographs of moderate clustering of gold nanoparticles embedded in a polymerized shape memory polymer at 1 wt % (FIG. 4D) while FIG. 4E is a polymerized shape memory polymer at 0.5 wt %.

FIGS. 5A-5E are a series of plots charting thermo-mechanical properties of gold nanocomposite materials, as measured by dynamic mechanical analysis, presented in the following order: glass transition temperature and transition width (FIG. A); glassy modulus (FIG. B); rubbery modulus (FIG. C); free strain recovery and strain fixity (FIG. D); and shape recovery sharpness (FIG. E).

FIGS. 6A-6D are a series of schematic, perspective views of a photopolymerized SMP film containing DDT-functionalized GNPs, displaying recovery of its permanent shape, following deformation at 50° C.

FIGS. 7A and 7B provide representative storage modulus and tan delta curves for shape memory polymers with and without gold nanoparticles.

FIGS. 8A-8C is a series of graphs setting forth uniaxial tensile behavior of shape memory polymer—gold nanocomposites: tensile modulus (FIG. A); strain at break (FIG. B); and peak stress (FIG. C).

FIG. 9 graphically illustrates UV-Vis absorption spectrum of SMP-GNP composite films containing various gold nanoparticle concentrations (with 10 mg/ml ˜1 wt %).

FIGS. 10A-10F are a series of top, isometric schematic views that together show the shape recovery of SMP-GNP composite strip after temporary deformation (rolling) upon illumination by a 100 mW, 532 nm (nominally green) solid state laser.

FIG. 11 is graphic representation of infrared spectra of an unreacted acrylate monomer film and two polymerized nanocomposite materials containing 0 and 1 wt % gold nanoparticles.

FIG. 12 is a perspective view illustrating one embodiment of a closure device for transcatheter operations, which can be made with a GNP nanocomposite according to the present disclosure.

FIG. 13 is a side view of an embolic coil, which can be made with a GNP nanocomposite according to the present disclosure.

FIG. 14 is a side view of an embodiment of a temporary venous filter system (TVFS) that generally includes a catheter and a venous filter, with the filter being able to be made with a GNP nanocomposite according to the present disclosure.

FIG. 15 is a side view of one embodiment of a vascular graft, which can be made with a GNP nanocomposite according to the present disclosure.

FIG. 16 is a side, perspective view of one embodiment of a septal defect closure device, which can be made with a GNP nanocomposite according to the present disclosure.

FIG. 17 is top perspective view of one embodiment of a stent, which can be made with a GNP nanocomposite according to the present disclosure.

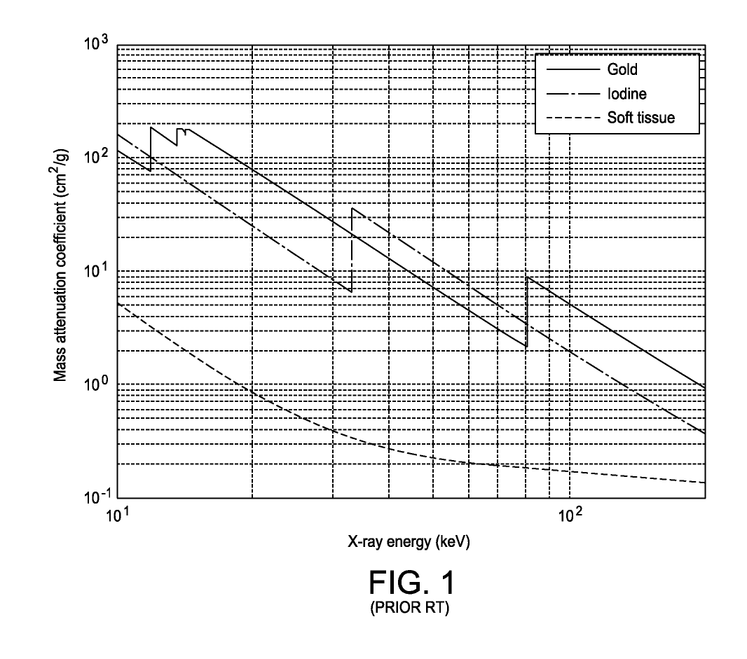

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

While incorporation of GNPs represents a promising avenue for enhancement of X-ray contrast and mechanical properties in SMPs for trans-catheter device applications, several synthesis-related challenges are considered to exist. For instance, it is well known that nanoparticles tend to aggregate when placed in a dissimilar environment, but also that generating a well-dispersed composite is generally necessary to ensure reproducible results and to help avoid failures at the interface between the polymer and additive. Salamone J., Radiopaque Polymers. Polymeric materials encyclopedia: Q-S. 1996:7346-7350] Synthesizing a monodisperse composite containing high nanoparticle concentrations may advantageously employ surface-functionalization of GNPs to improve their miscibility, precise regulation of particle size and polymer composition, adequate energy input for dispersion, and/or control of SMP polymerization kinetics. To simplify this process, a photo-polymerized acrylate SMP may be used as a starting material. It, in one form thereof, consists of 80 wt % tert-Butyl acrylate (tBA) and 20 wt % poly(ethylene glycol)dimethacrylate (PEGDMA). tBA forms the backbone of the polymer and confers significant hydrophobicity, while PEGDMA acts as a cross-linker. This formulation and acrylates, in general, are well-characterized and yield highly inert, optically clear polymers, with excellent oxidative/thermal stability, minimal tissue response, and no MRI or CT artifact. Small W, Singhal P, Wilson T S, Maitland D J., Biomedical applications of thermally activated shape memory polymers. Journal of materials chemistry. 2010;20(18):3356-3366; Gall K, Yakacki C M, Liu Y, et al., Thermomechanics of the shape memory effect in polymers for biomedical applications. Journal of biomedical materials research. Part A. 2005;73(3):339-48. Available at: http://www.ncbi.nlm.nih.gov/pubmed/15806564. Accessed Aug. 9, 2010; Yakacki C M, Shandas R, Safranski D, et al., Strong, Tailored, Biocompatible Shape-Memory Polymer Networks. Advanced functional materials. 2008;18(16):2428-2435. Available at: http://www.pubmedcentral.nih.gov/articlerender.fcgi?artid=2714647&tool=pmcentrez&rendertype=abstract. Accessed Jul. 26, 2010]

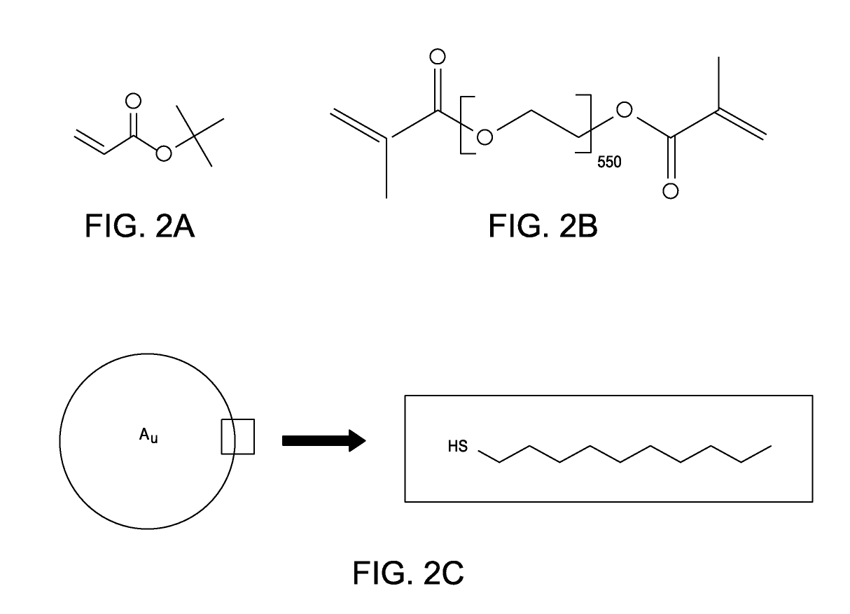

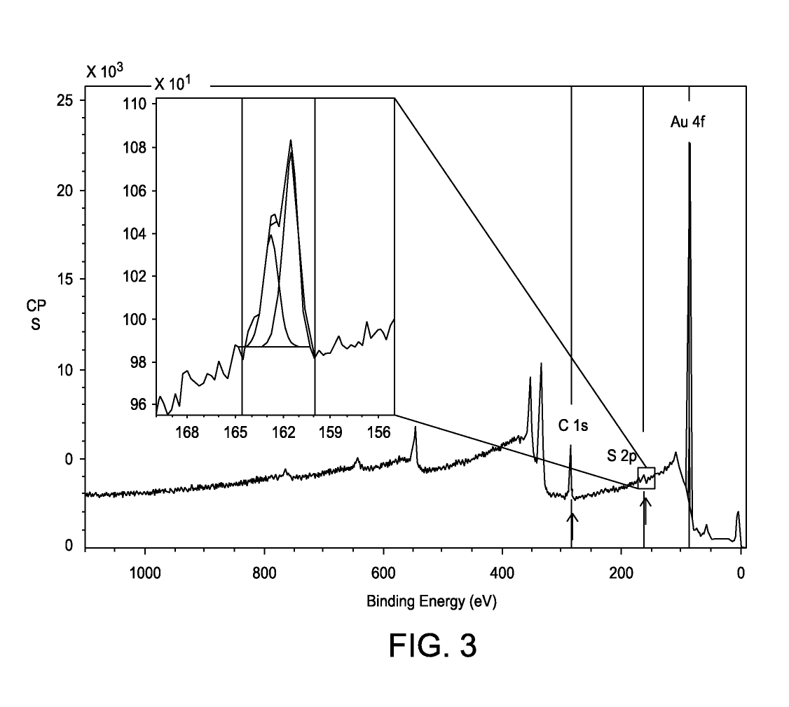

These two monomer compounds, along with a schematic of the cross-linking, are shown in FIGS. 2A-2C. In particular, per FIG. 2, the general chemical structure of monomers used: tert-Butyl acrylate (tBA) (FIG. A); and poly(ethylene glycol)dimethacrylate (PEGDMA) 550 Mn is shown (FIG. B), while cross-linking with a gold nanoparticle is set forth in FIG. 2C. Additionally, FIG. 3 shows the X-ray photoelectron spectrum of a DDT-functionalized gold nanoparticle surface formed in accordance with the present system. The binding energy associated with the sulfur 2 p peak shows successful thiolation.

Maximizing radio-opacity can simply be a matter of determining which modifiable nanoparticle and polymer variables maximize the mass of gold that can be incorporated into the SMP per unit volume. However, it is important to remember that all final composite material properties can be highly interdependent. For instance, increasing the concentration of GNPs will typically enhance X-ray contrast, but may raise the rubbery modulus (ie. stiffness above the T_{g}) of the composite, possibly to undesirable levels for a particular application. As a result, it can be important to simultaneously consider whether the resulting composite material also displays similar thermo-mechanical properties that make the original SMP an attractive candidate for TCD fabrication. These properties may include, for example, a T_{g} close to body-temperature, a low elastic modulus in the rubbery state, and/or high strain recovery and strain fixity rates (the abilities to recover permanent and store temporary shapes, respectively). Achieving these goals may employ hypothesis-driven manipulation of several available variables, such as those provided in Table 1.

TABLE 1
Modifiable nanoparticle and polymer variables and their
expected effect on final composite material properties.

| Variable | Examples | Composite property affected |
|---|---|---|
| Nanoparticle | | |
| Concentration | 0 → 10 wt % range | Radio-opacity, MRI/CT artifact, |
| | | Tg, shrinkage stress, strain- |
| | | recovery, rubbery and glassy |
| | | moduli |
| Size and aspect | 5-100 nm diameter range, | Optical absorption properties, |
| ratio | spherical vs. rod-shaped | concentration, monodispersity |
| Surface-modifier | Alkanethiols, methyl-PEG- | Stability, concentration limit, |
| | thiol** compounds, 11- | monodispersity |
| | Mercaptoundecyl- | |
| | tetra(ethylene glycol), 1- | |
| | mercapto-(triethylene glycol) | |
| | methyl ether | |
| Method of | Ex situ production and | Concentration limit, |
| nanoparticle | incorporation, In situ | monodispersity |
| incorporation | reduction of metal salts, | |
| | pyrolysis of metal-containing | |
| | precursors | |
| Polymer | | |
| Type/functionality | Acrylic acids, acrylic salts, | Tg, strain-recovery, rubbery |
| and stoichiometry of | methacrylates, metal- | and glassy moduli |
| monomers | chelating side groups | |
| Cross-linking | | Tg, strain-recovery, rubbery |
| monomer molecular | | and glassy moduli |
| weight and density | | |
| Polymerization | UV irradiation, thermal, redox | Concentration limit, tensile |
| method | | strength, Tg, rubbery and |
| | | glassy moduli |

**Methyl-PEG-thiol: A molecule consisting of poly(ethylene glycol) terminated at either end with a methyl group and thiol

Nanoparticle Variables

Like most metal nanoparticles, GNPs may be synthesized chemically by reacting a metal salt precursor, such as hydrogen tetrachloroaurate (HAuCl₄), with a strong reducing agent, at elevated temperature. To help ensure that gold metal forms in distinct particles, this reaction typically requires a stabilizing component, such as a short polymer. Daniel M-C, Astruc D., Gold nanoparticles: assembly, supramolecular chemistry, quantum-size-related properties, and applications toward biology, catalysis, and nanotechnology. Chemical reviews. 2004;104(1):293-346. Available at: http://www.ncbi.nlm.nih.gov/pubmed/14719978; Philip D., Synthesis and spectroscopic characterization of gold nanoparticles. Spectrochimica acta. Part A, Molecular and biomolecular spectroscopy. 2008;71(1):80-5. Available at: http://www.ncbi.nlm.nih.gov/pubmed/18155956; Chan, W C W ed. Bio-applications of nanoparticles. Austin: Landes Bioscience; 2007] The stabilizing component acts as a temporary surface ligand, preventing aggregation of particles and regulating their size. In general, higher initial gold salt concentrations, stronger or more concentrated reducing agents, higher capping ligand concentration, and/or shorter reaction times all tend to yield smaller particles. Chan W C W ed., Bio-applications of nanoparticles. Austin: Landes Bioscience; 2007.

Particles can be formed using an Ex situ or In situ approach. In situ formation involves reducing the gold precursor in the liquid monomer mixture (tBA and PEGDMA) prior to polymerization/curing. The Ex situ approach, in which particles are generated separately, added to the liquid monomer mixture, and dispersed with sonication, simplifies the removal of unwanted reactants and reaction byproducts. While the Ex situ approach was chosen, given its simplicity, to generate test samples with respect to the present embodiments, it is to be understood that both Ex Situ and In situ approaches are valid means of particle formation and, thus, within the scope of this disclosure. Once formed, the temporary surface passivating agent can be replaced by reacting the GNPs with any molecule terminated by a chemical group with higher affinity for the gold surface. These groups include thiols, amines, and phosphines. Ultimately, the GNPs generally take on the character (e.g., hydrophobicity) of this secondary surface ligand.

Nanoparticle size may be assessed through transmission or scanning electron microscopy (TEM or SEM, respectively) or using spectroscopic techniques, such as dynamic light scattering (DLS) and/or UV-Vis spectroscopy. Spectroscopic techniques provide rapid results, but these techniques rely on the application of the Mie and Gans theories for spherical particles and can be prone to minor inaccuracies. Haiss W, Thanh N T K, Aveyard J, Fernig D G., Determination of size and concentration of gold nanoparticles from UV-vis spectra. Analytical chemistry. 2007;79(11):4215-21. Available at: http://www.ncbi.nlm.nih.gov/pubmed/17458937; Amendola V, Meneghetti M., Size Evaluation of Gold Nanoparticles by UV-vis Spectroscopy. The Journal of Physical Chemistry C. 2009;113(11):4277-4285. Available at: http://pubs.acs.org/doi/abs/10.1021/jp8082425] Once incorporated into the cured SMP, monodispersity can be accurately assessed with electron microscopy techniques. Aggregation is generally measured in terms of the average GNP cluster size.

It is expected that smaller GNPs will reduce the entropic penalty associated with their incorporation into the continuous phase of the polymer and thereby disperse more readily. It is also expected that miscibility of the GNPs with the SMP may be optimized by matching the hydrophobicity of chemical groups on the gold surface to that of the constituent monomers. Since polymers display local variations in hydrophobicity, it is predicted that a surface “brush” which is heterogeneous in both hydrophobicity and size can optimize dispersion and the capacity of the SMP matrix to support GNPs. Accordingly, GNPs with varying ratios of hydrophobic and hydrophilic/amphiphilic surface ligands and ligands with varying molecular weights can be generated, with their solubility limits in the monomer mixture then determined. Dodecanethiol (hydrophobic) has been successfully used as a surface ligand. Other options include 11-Mercaptoundecyl-tetra(ethylene glycol) (hydrophilic), 1-mercapto-(triethylene glycol)methyl ether (amphiphilic), and variations of these ligands with fewer or more repeating units. In other words, heterogeneity in ligand molecular weight could also be explored, with the goal of improving GNP dispersion and incorporable mass. Attention will have to be paid to the molecular weight cut-off of the surface modifiers, as large molecular weight tags may strongly influence the physical properties of the nanoparticles and the resulting composite material.

Polymer Variables

Cross-linked, acrylate SMPs are generally synthesized by injecting a mixture of acrylate monomers into a mold, in the presence of a photo-cleavable initiator species, and exposing the mold to UV irradiation until a high degree of bond conversion is achieved. Composite SMPs may initially be synthesized using the GNP concentration at an upper determined limit and the associated surface chemistry. A mixture of 80 wt % tBA and 20 wt % PEGDMA containing the resuspended GNPs, in one variation, can be injected into rectangular glass molds which includes a 1 mm rubber spacer sandwiched between two glass slides. These molds may be exposed to a 20 mW/cm² UV source until

Dispersion of the GNPs in the acrylate mixture for thermal polymerization was achieved by sonicating the mixtures in an ice water bath for 2 hours, after which the temperature of the bath was ramped up to 70° C. The time required for polymerization following the temperature ramp depends on the type and concentration of thermal initiator used. A typical preparation utilizes 0.15% (w/w) 2,2′-Azobis(2-methylpropionitrile) (AIBN). Cross-linker molecular weight was selected among numerous modifiable polymer variables due to its expected influence on matrix mobility and hence, GNP incorporation. Longer cross-linkers should accommodate GNPs more readily, making aggregation energetically unfavorable. Its effect maybe measured, in one manner, by substituting three different species of PEGDMA into the base formulation, having number-average molecular weights of 550, 750 and 1000. As before, aggregation tendency can be measured via TEM examination of the average GNP cluster size.

It is anticipated that GNPs may alter polymerization kinetics at high concentrations by interfering with UV radiation and generating excessive heat in the process. These combined effects may extend the time necessary to achieve high rates of bond conversion, allowing GNPs previously dispersed by sonication to aggregate before complete gelation of the polymer. As a result, UV-initiation can be compared to non-irradiative initiation techniques (thermal and redox initiation). This UV-initiation technique involves, in part, the replacement of the photo-cleavable initiator species with thermal or redox initiator molecules. The use of sonication to maintain particle dispersion during polymerization is a process variable for consideration, as well.

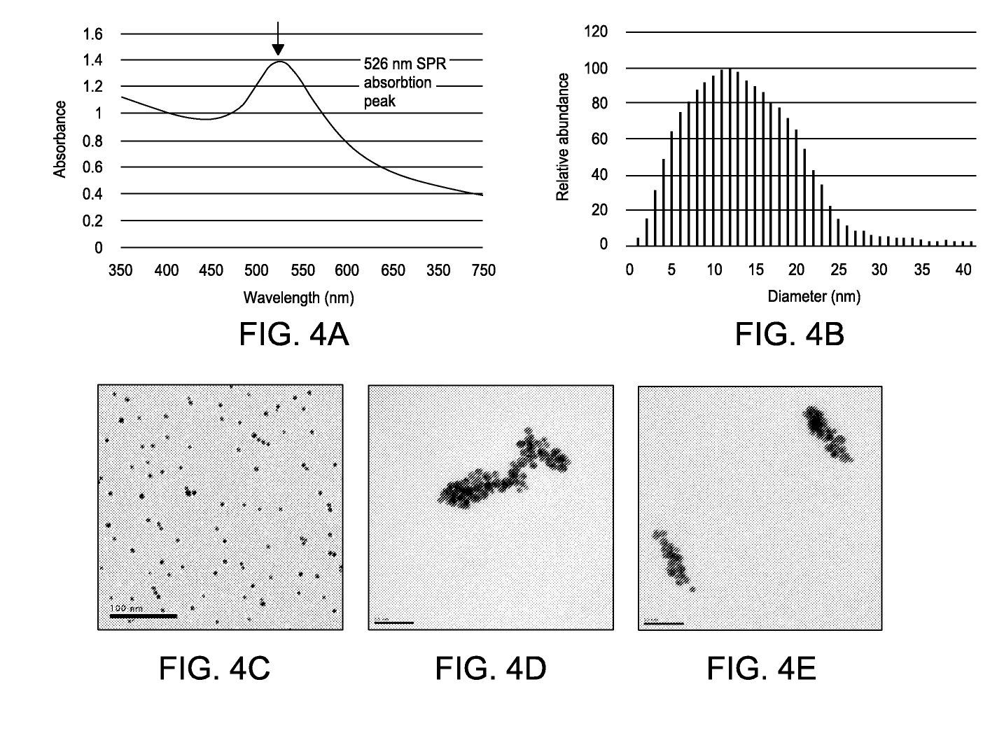

FIGS. 4A-4E together help to characterize DDT-functionalized gold nanoparticles. UV-Vis spectrum (FIG. 4A) and dynamic light scattering data (FIG. 4B) of nanoparticles indicate average particle sizes of approximately 12 and 14 nm, respectively. Additionally, the representative UV-Vis absorption spectrum of ˜10 nm GNPs in aqueous environment showing SPR peak in the range of 520-530 nm and, more particularly, at 522 nm. Meanwhile, transmission electron micrographs of DDT-functionalized gold nanoparticles show excellent dispersion in purely hydrophobic environments like hexane (FIG. 4C) and moderate clustering when embedded in a polymerized shape memory polymer at 1 wt % (FIGS. 4D, 4E).

Assessing Mechanical and Thermo-Mechanical Properties

Dynamic mechanical analysis (DMA) may be used to assess the effect of GNP concentration on final composite thermo-mechanical properties. Essentially, a series of thin-film SMP-GNP composites, in one embodiment, may be generated with GNP concentrations ranging from 0 wt % to the highest desired concentration. These films may be loaded between DMA clamps for thermal scans, running between 0 and 100° C.

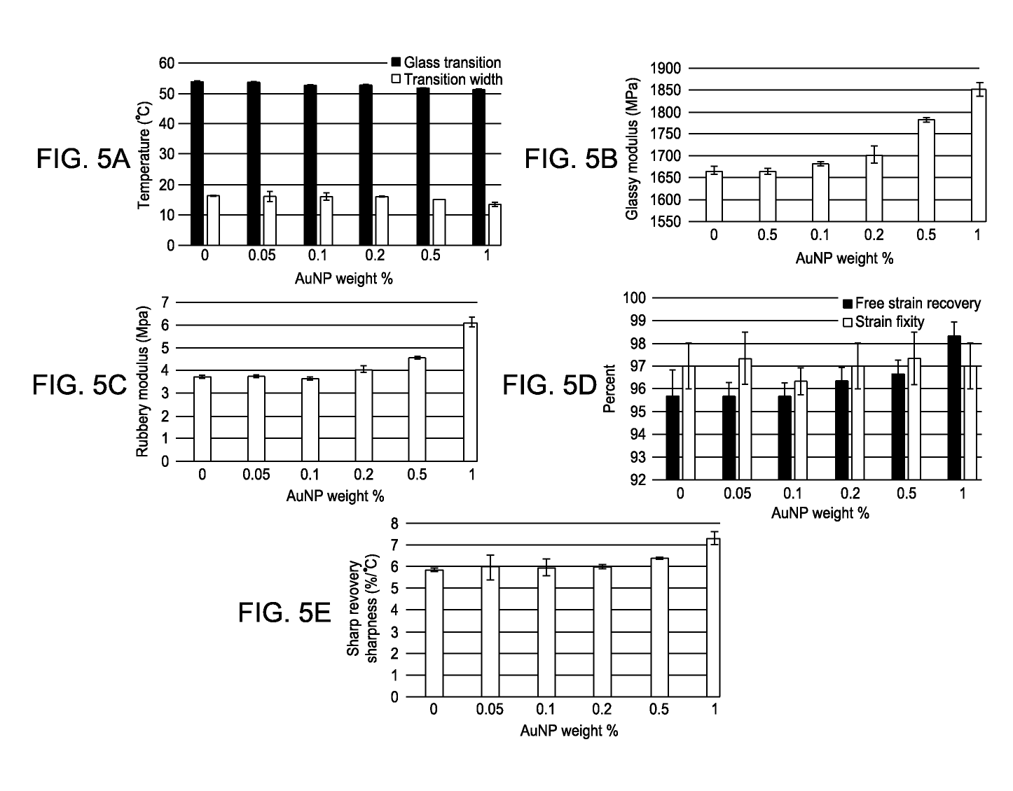

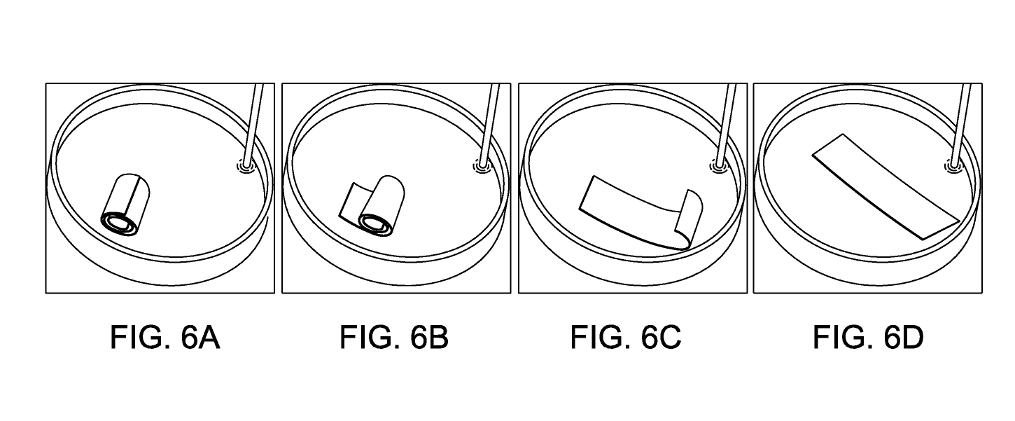

Glassy and rubbery moduli, glass transition temperature, free strain recovery and fixity rates, and shape recovery sharpness may be monitored and observed (FIGS. 5A-5E). In particular, those thermo-mechanical properties of nanocomposite materials, as measured by dynamic mechanical analysis, are portrayed within FIG. 5, in the following order: glass transition temperature and transition width (FIG. A); glassy modulus (FIG. B); rubbery modulus (FIG. C); free strain recovery and strain fixity (FIG. D); and shape recovery sharpness (FIG. E). It is expected that at low concentrations, GNPs act as plasticizers, separating polymer chains, reducing crystallinity in the SMP, and lowering moduli and thermal transitions. However, at high concentrations, closer to the percolation threshold of the composite, it is expected that the properties of the GNPs themselves, namely high moduli, may be expected to emerge.

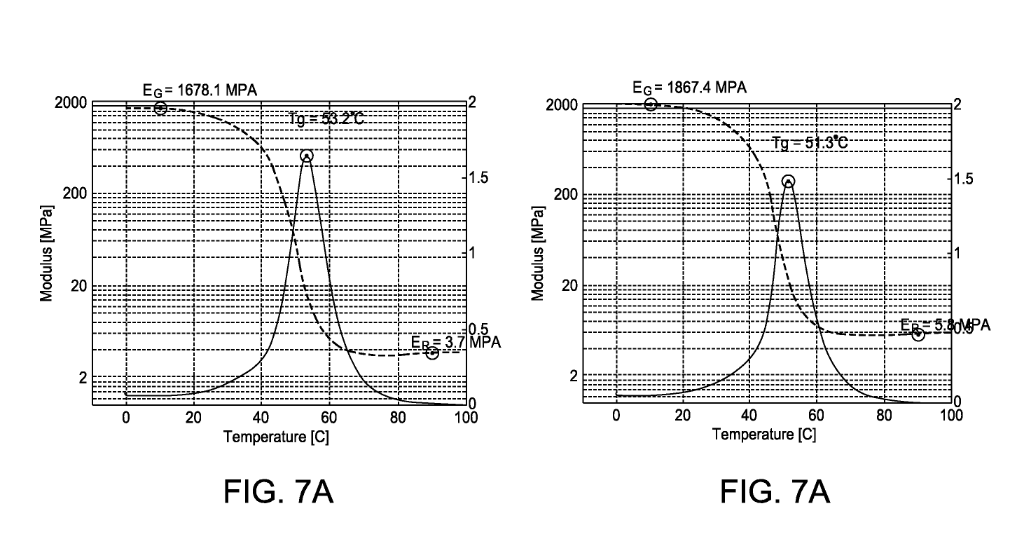

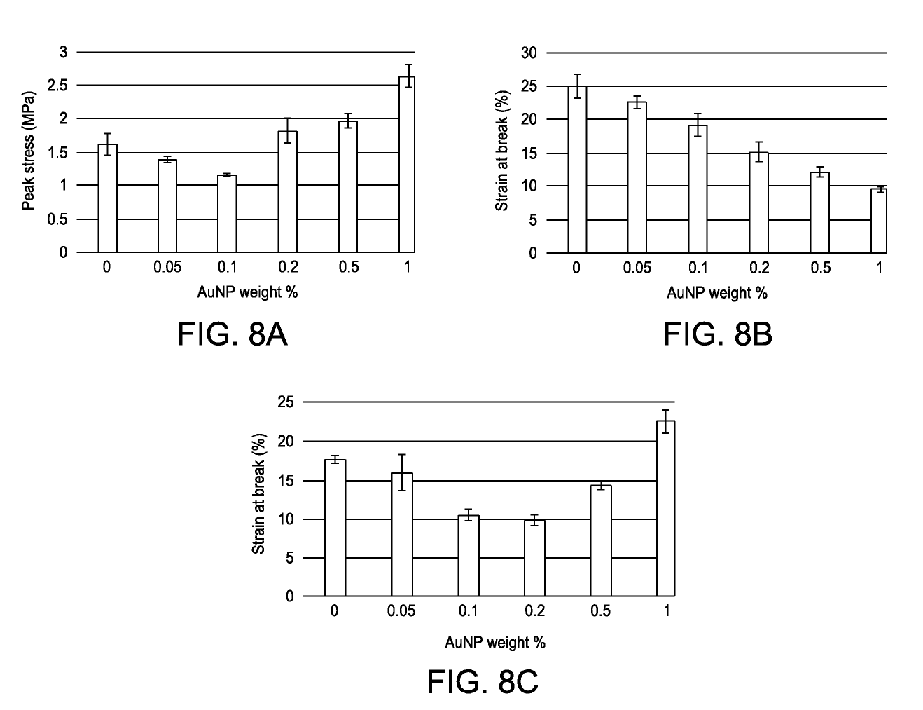

Further data, both visual and graphical, is available with respect to the mechanical properties of the present GNP polymeric materials. FIGS. 6A-6D show an example of the effects of how, for example, such free strain recovery and fixity rates, and shape recovery sharpness in play in an actual test component. In particular, those drawings together schematically simulate recovery of the permanent shape of a photopolymerized SMP film 20 containing DDT-functionalized GNPs, following deformation at 50° C. FIGS. 7A and 7B provide representative storage modulus and tan delta curves for shape memory polymers with and without gold nanoparticles. Meanwhile, FIGS. 8A-8C) sets forth uniaxial tensile behavior of shape memory polymer—gold nanocomposites: tensile modulus (FIG. 8A); strain at break (FIG. 8B); and peak stress (FIG. 8C).

Electrical and thermal conductivity may be measured, for example, by four-terminal sensing and transient plane source sensors, respectively. Both types of conductivity are expected to increase as a function of GNP concentration because native polymers are electrical and thermal insulators, whereas gold is an excellent conductor of electrons and heat. The most dramatic change is expected near the percolation threshold of the composite, when the discontinuous phase (GNPs) begin to form a continuous connected network within the polymer.

Accelerated oxidation tests may be to evaluate the resistance of nanocomposite samples to oxidative degradation. Briefly, 1 g thin-film samples of SMP-GNP composites (over a range of GNP concentrations), in one version of such a test, may be exposed to 30 v/v % hydrogen peroxide solutions at 37° C. for a period of one month (simulating approximately 2 years of direct blood contact). Rocha M F G, Mansur A a P, Martins C P S, Barbosa-Stancioli E F, Mansur H S., Macrophage Response to UHMWPE Submitted to Accelerated Ageing in Hydrogen Peroxide. The open biomedical engineering journal. 2010;4:107-12. Available at: http://www.pubmedcentral.nih.gov/articlerender.fcgi?artid=2923342&tool=pmcentrez&rendertype=abstract.] Solutions are typically replaced every 5 days to maintain activity, given the anticipated 7-day activity half-life of hydrogen peroxide at 37° C. It is expected that composite samples with higher concentrations of gold nanoparticles will exhibit lower rates of mass loss over time, due to the known free-radical scavenging activity of GNPs.

Assessing Imaging and Other Special Properties

Using a similar series of thin-film SMP-GNP composite samples as generated for earlier mechanical and thermo-mechanical analysis influence of GNP concentration on X-ray contrast, optical transduction, and MRI signal may be assessed. A digital, voltage-controlled fluoroscopic scanner and 3T MRI scanner can be used to determine linear X-ray attenuation coefficients and MR signal/artifact generation for nanocomposite samples. All imaging may be performed, in one test scenario, in a custom-designed imaging phantom, which can include a water-tight, open acrylic box and a monofilament nylon wire (not shown). Samples may be suspended with the monofilament nylon wire at the center of the box, within a 6-inch column of normal saline. The saline mimics the X-ray attenuation/scattering properties of most biological tissues; for MRI studies, 5 mM gadolinium chelate contrast may be added in order to reduce the T1 relaxation time to appropriate levels. Radio-opacity is expected to increase linearly with GNP content. Polymers are generally visible during MRI scans through negative contrast, since the relaxation of protons in the surrounding water generates signal. Since gold is a diamagnetic element and should not act as a source of magnetic field inhomogeneity, no change is expected in MRI signal and SMP-GNP nanocomposites should not generate MRI artifacts.

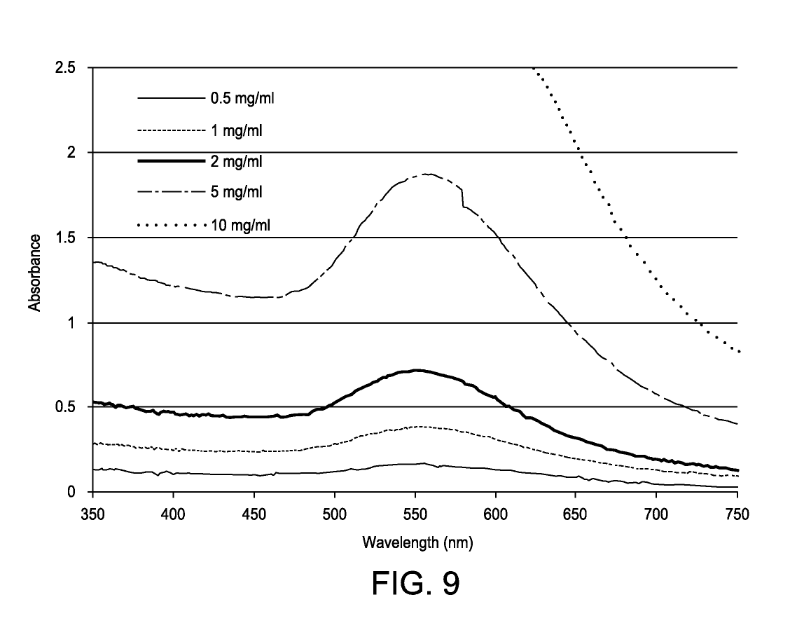

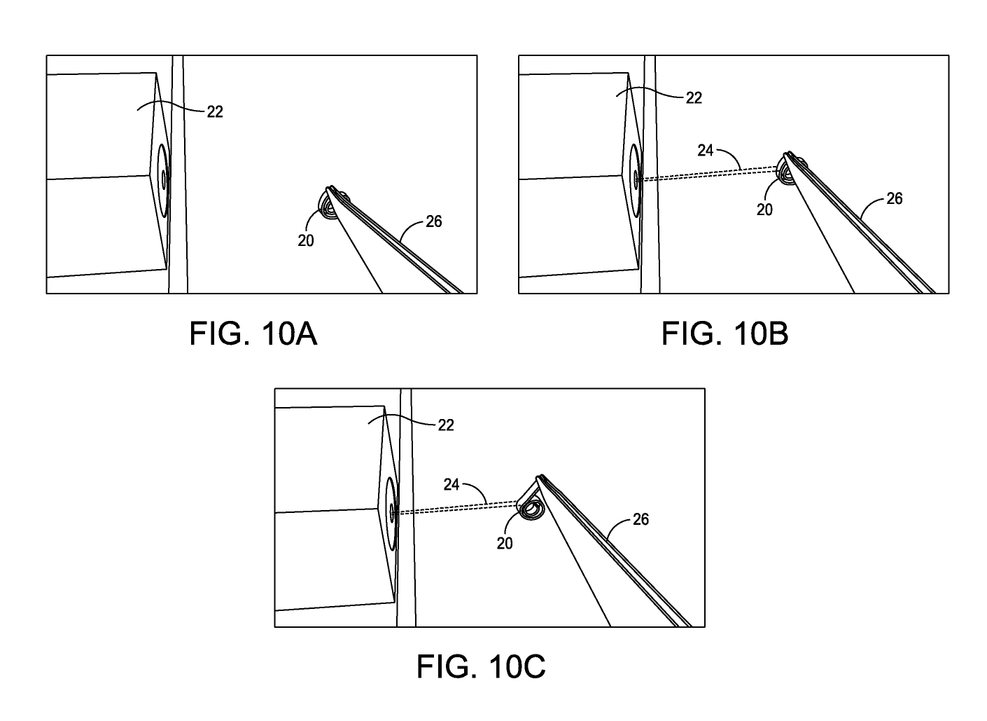

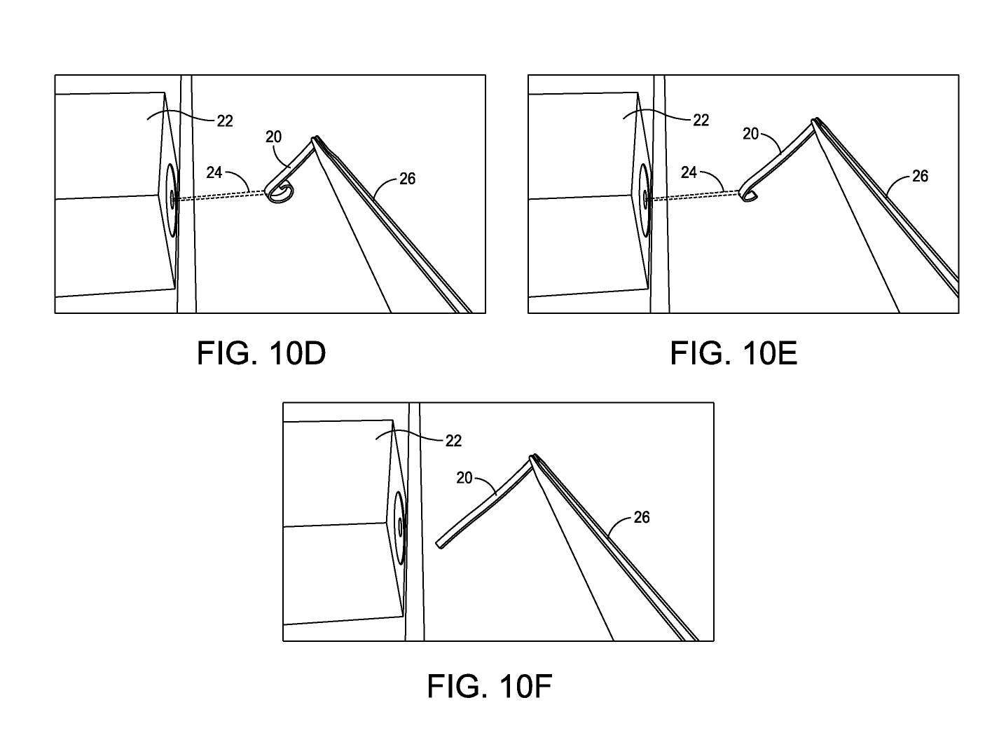

Further, GNPs are known to exhibit a characteristic absorption peak associated with the surface plasmon resonance (SPR) phenomenon. Chan, W C W ed. Bio-applications of nanoparticles. Austin: Landes Bioscience; 2007; 41. Wang Z, Ma L. Gold nanoparticle probes. Coordination Chemistry Reviews. 2009;253(11-12):1607-1618. Available at: http://linkinghub.elsevier.com/retrieve/pii/S001085450900006X. Accessed Mar. 26, 2011; Zijlstra P, Bullen C, Chon J W M, Gu M., High-temperature seedless synthesis of gold nanorods. The journal of physical chemistry. B. 2006;110(39):19315-8. Available at: http://www.ncbi.nlm.nih.gov/pubmed/17004786. Most GNPs exhibit a SPR-enhanced absorption peak between 520 and 530 nm, which falls in the green portion of the visible light spectrum. FIG. 9, which graphically illustrates UV-Vis absorption spectrum of SMP-GNP composite films containing various gold nanoparticle concentrations (with 10 mg/ml ˜1 wt %), indicates that such an absorption peak range is, at least at concentrations of 1 wt % or less, essentially independent of the specific GNP concentration employed. It is thus expected that GNPs will absorb and dissipate green light as heat very efficiently, allowing an indirect and spatially-controllable method of triggering thermal transitions in the composites. Heat dissipation may, in one test embodiment, be measured using a differential scanning calorimeter with an integrated fiber-optic line and monochromatic, collimated light source. This ability to absorb and dissipate green laser light is schematically illustrated in FIGS. 10A-10F. The views provided together show the shape recovery of an SMP-GNP composite strip 20 after temporary deformation (rolling) upon illumination by 100 mW, 532 nm (nominally green) solid state laser beam 22, generated by an appropriate laser 24. The SMP-GNP composite strip 20 is held during testing using a clamp device 26, which could, for the purposes of this test, could be as simple as a pair of tweezers (such as shown in the illustrated test embodiment). Per the example schematically illustrated in FIGS. 10A-10F, the GNP-polymer, estimating a specific heat of 1.5 J/g-K, it is expected that it will take 15 sec to raise the temperature of 1 g of the material by 1° C.

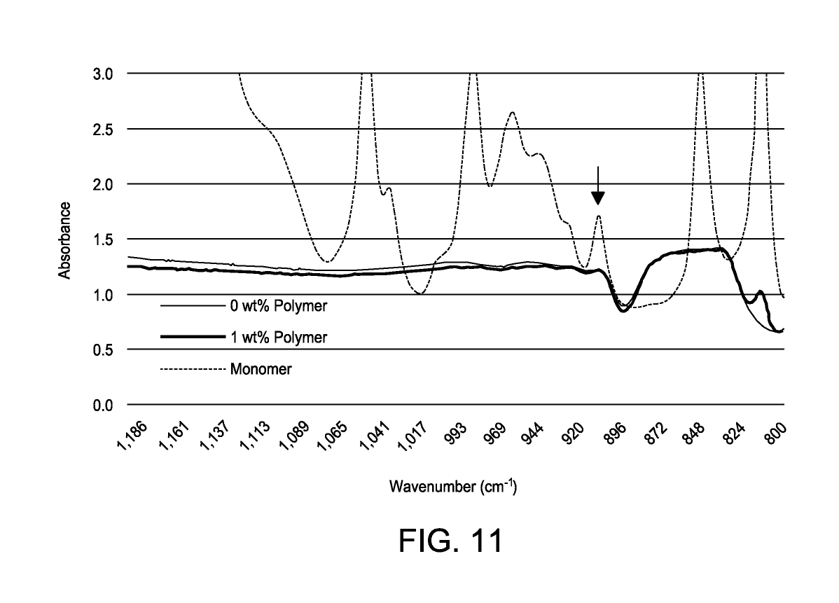

A potential area of concern is whether the GNP concentration would have any deleterious effect on polymerization/bond conversion. FIG. 11 graphically shows infrared spectra of an unreacted acrylate monomer film and two polymerized nanocomposite materials containing 0 and 1 wt % gold nanoparticles. The absence of a vinyl group absorbance peak at 900 cm⁻¹ shows adequate bond conversion, independent of nanoparticle concentration. As such, for the GNP concentrations of interest, sufficient polymerization/bond conversion is not an issue for this material.

As mentioned above, one of the limitations of this study associated with the present application is that only a limited number of the modifiable nanoparticle and polymer variables listed in Table 1 were evaluated. As a result, it may underestimate the true range in final composite properties that may be achievable through similar methods. In addition, it is unlikely that all of the optimal properties for an individual TCD application may be found at a single GNP concentration. Instead, the results should be compared to current designs for superiority with regard to the most important material characteristics and cost. In some cases, radio-opacity will have to be balanced with desired mechanical properties. It is thus to be understood that other combinations of materials and/or process parameters, as known in the art, may be employed, as desired, to arrive at a desired GNP polymeric material for use in a given TCD application, and such process variations are deemed to be within the scope of the present system. Further, while it is clear that an entire TCD part could be made entirely of a desired GNP polymeric material, it is to be understood that, in some instances, it may prove beneficial to provide, e.g., a core layer and/or an outer film of such a GNP polymeric material, with the remainder being made of another desired material, e.g., a shape-memory polymer, as sufficient to achieve the desired radio-opacity and mechanical properties for a given part, and such a constructed TCD is considered to be within the scope of the present system.

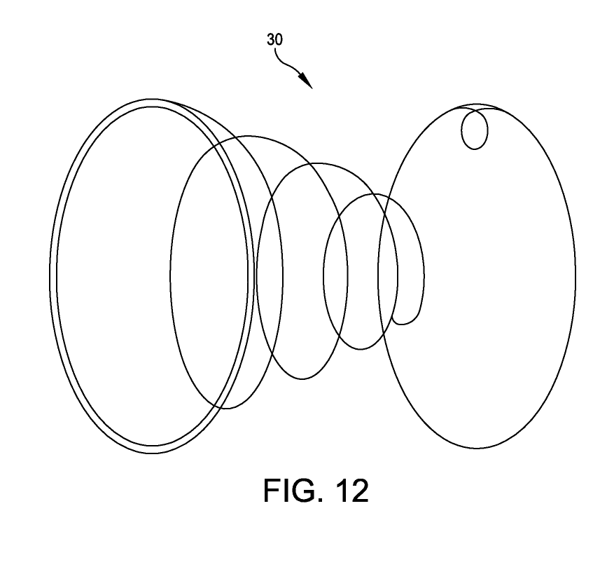

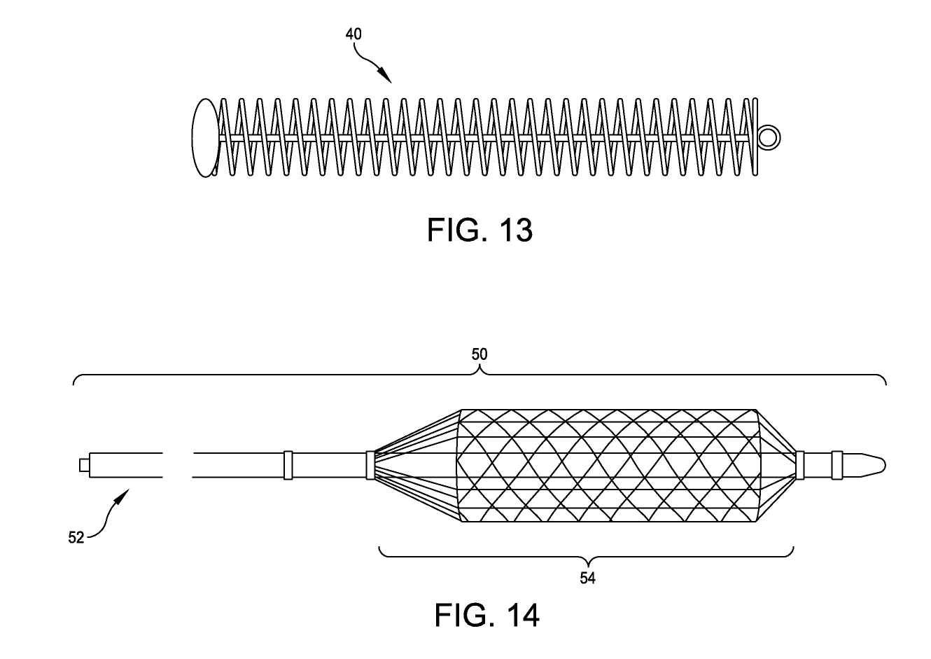

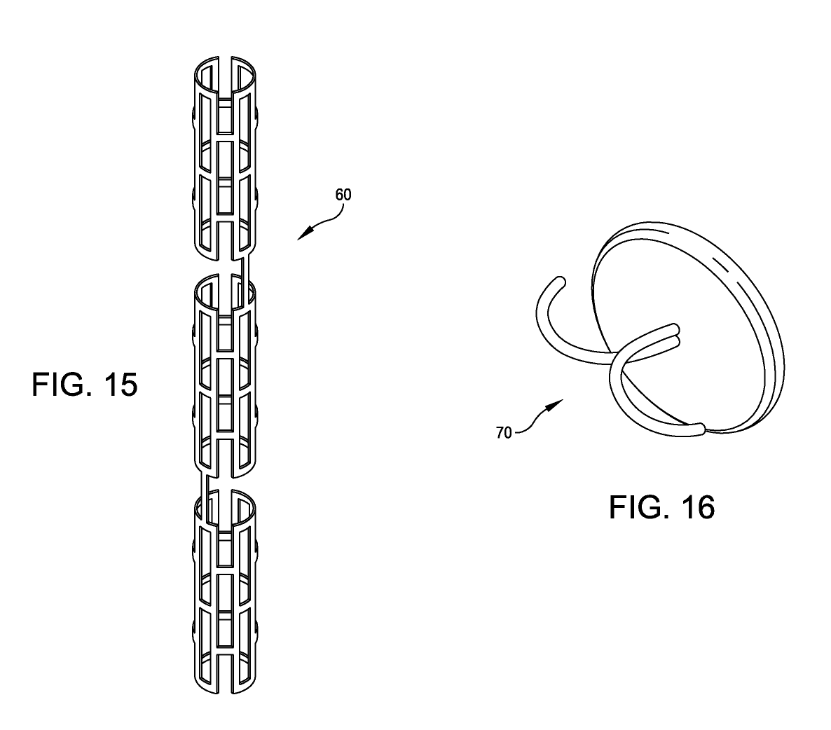

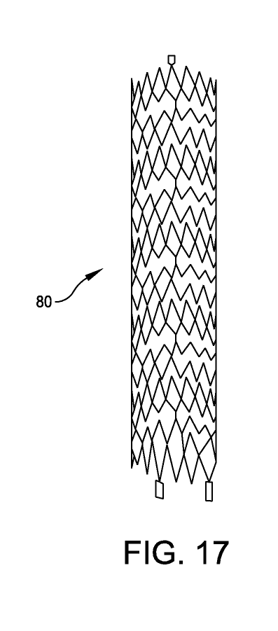

FIGS. 12-17 illustrate, for sake of example only, various known TCDs in which the novel GNP polymeric material of the present application may now be employed. It is to be understood that other TCDs not shown could also employ such a material, and such TCDs would also be considered within the scope of this disclosure. FIG. 12 illustrates is a perspective view illustrating one embodiment of a closure device 30 for transcatheter operations, constructed as set forth in U.S. Pat. No. 6,375,671 B1. FIG. 13 shows an exemplary embolic coil 40, such as that provided in US2014018844 (A1). FIG. 14 depicts a temporary venous filter system 50 (TVFS) that generally includes a catheter 52 and a venous filter 54, such as that illustrated in WO2010132173 (A1), with the filter 54, per the present invention, being able to be made of the GNP-polymer composite described herein. FIG. 15 is a side view of one embodiment of a vascular graft, as set forth by U.S. Pat. No. 5,902,332, and, by extension of the present inventive concept, could be made of a GNP-polymer composite. FIG. 16 illustrates an embodiment of a septal defect closure device (per US2012065673), which can be made with a GNP nanocomposite according to the present disclosure. FIG. 17 shows an embodiment of a stent (such as one shown on Wikipedia, as accessed Sep. 17, 2014), which can be made with a GNP nanocomposite according to the present disclosure.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A trans-catheter cardiovascular device, comprising a composite material having gold nanoparticles embedded in a shape memory polymer.

2. The device of claim 1 wherein the gold nanoparticles are surface-functionalized gold nanoparticles.

3. The device of claim 2 wherein the gold nanoparticles comprise about 10 wt % or less of the composite material.

4. The device of claim 2 wherein the composite includes gold nanoparticles in the size range of <100 nm.

5. The device of claim 1 wherein the shape memory polymer is a cross-linked shape memory polymer.

6. The device of claim 3 wherein the shape memory polymer is formed by cross-linking at least one of an acrylate, an acrylic acid, an acrylic salts, a methacrylate, and a metal-chelating side group.

7. The device of claim 1 wherein the shape memory polymer forms one of a stent, an embolic coil, a venous filter, a vascular graft, and a cardiac septal defect closure device.

8. The device of claim 1 wherein gold nanoparticles with a size of approximately 10 nm display a UV-visible light absorption peak in the range of 500-550 nm.

9. The device of claim 1 wherein the gold nanoparticles are present in an amount sufficient to promote radio-opacity of the composite.
